(19) United States
(12) Patent Application Publication
(10) Pub. No.: US 2005/0013858 A1
(43) Pub. Date: Jan. 20, 2005

(54) Oral pharmaceutical compositions containing terbinafine

(76) Inventors: Stefan Hirsch, Lorrach (DE); Friedrich Karl Mayer, Oberwil (CH); Carle Paul, Mulhouse (FR); Patrice Guitard, Hegenheim (FR)

Correspondence Address: NOVARTIS;CORPORATE INTELLECTUAL PROPERTY, ONE HEALTH PLAZA 430/2, EAST HANOVER, NJ, 07936-1080, US

(21) Appl. No.: 10/311,177

(22) PCT Filed: Jun. 11, 2001
(86) PCT No.: PCT/EP01/06596

(51) Int. Cl. A61K 9/26; A61K 31/137

(52) U.S. Cl. 424/469; 514/649

(57) ABSTRACT

The present invention relates to sustained release pharmaceutical composition for oral administration comprising terbinafine.

This invention relates to terbinafine pharmaceutical compositions, and the use of such compositions.

Terbinafine is an orally effective anti-fungal agent, available under the registered trademark Lamisil. It is effective in a wide range of fungal infections. Terbinafine is particularly useful against dermatophytes, contagious fungi that invade dead tissues of the skin or its appendages such as stratum corneum, nails, and hair. Such a nail fungus makes its home in the nail bed, shielded by the hard outer nail, Thus once the infection is established under the nail, the nail itself provides the fungus with a protected environment that allows it to grow. The effects of these fungi on the nails may be unsightly, seriously complicate foot-care, have a deleterious impact on patients' overall quality of life, and well-being and impair the patients' ability to work. If left untreated, the fungi can deform toe-nails permanently and lead to pain on walking. Additionally the fungi can lead to fissures in the skin encouraging bacterial infections. Serious complications as a result of these infections may occur in people suffering from diabetes such as diabetic foot syndrome including primary disease-related complications, e.g gangrene, that, ultimately, can be life-threatening or require amputations. Other high-risk patient sub-groups include patients infected with human immunodeficiency virus (HIV), patients with acquired immunodeficiency syndrome (AIDS), and patients with other types of immunosuppression (e.g, transplant recipients and patients on long-term corticosteroid therapy). There is an increased prevalence of onychomycosis in the elderly (up to 30% by age 60). Microsporum, Trichophyton such as Trichophyton rubrum, or Trichophyton mentagrophytes and Epidermophyton such as Epidermophyton floccosum are those commonly involved. These infections are conveniently discussed according to the sites of the body involved. Diagnosis is confirmed by demonstrating the pathogenic fungus in scrapings of the lesions either by microscopic examination or by culture.

Across medical disciplines, onychomycosis is well recognized as being arduous both to diagnose and to manage particularly in the aged. Terbinafine is useful to treat toenail and fingernail onychomycosis due to dermatophytes (e.g. tinea unguium). Indeed terbinafine has opened up treatment for Tinea unguium caused by Tricloplyton. For example the Merck Manual of 1987 states that treatment of toe-nails should be discouraged with the previously used standard, Griseofulvin, because 1 to 2 years treatment is required, recurrence is usual and complete cure is unlikely.

For the onychomycosis use, terbinafine is normally administered as an immediate release tablet form containing 250 mg terbinafine once daily. Such a tablet sold under the name Lamisil releases terbinafine to the extent of 80% over a 30 minute period as measured by standard in vitro dissolution studies, e.g. at pH 3 using the paddle method. This is an example of an immediate release form. Terbinafine treatment over 12 weeks is required. The progress of its clinical effectiveness is seen with growth of the healthy nail, pushing out and replacing, the diseased unsightly nail containing debris and dead fungus. About 10 months is taken for a totally new toe-nail to form.

Although terbinafine is generally regarded as safe like any prescription drug, adverse events associated with its use have been reported. As described in the Physicians' Desk Reference, there have been a number of adverse events recorded, e.g. head-aches gastro-intestinal symptoms (including diarrhea, dyspepsia, abdominal pain, nausea, & flatulence), liver test abnormalities, e.g. enzyme abnormalities, dermatological symptoms such as pruritis, urticaria, rashes, and taste disturbances, e.g. loss of taste. These adverse events are in general mild and transient. Further adverse events include symptomatic idiosyncratic hepatobiliary dysfunction (e.g. cholestatic hepatitis), severe skin reactions such as Stevens-Johnson syndrome, neutropenia, and thrombocytopenia. Yet further adverse events may include and visual disturbances, such as changes in the ocular lens and retina as well as allergic reactions including anaphylaxis, fatigue, vomiting, arthralgia, myalgia and hair loss. Terbinafine is a potent inhibitor of CYP2D6 and may cause clinically significant interactions when co-administered with substrates of this isoform, such as nortriptyline, desipramine, perphenazine, metoprolol, encainide and propafenon. Hereinafter any and all these events are referred to as Adverse Events.

Some pharmacokinetic and biopharmaceutical properties of terbinafine are known. Thus terbinafine is well absorbed. Peak drug plasma concentrations (Cₘₐₓ) of about 1.3 microgram/milli-litre (with about a 20% variation e.g. 0.9 to 1.6 microgram/milli-litre) appear within 1 to 2 hours after administration of a single 250 mg terbinafine dose. The area under the curve over 24 hours (hereinafter AUC) is about 4.76 microgram.hour/millilitre. The increase in AUC is 42% when terbinafine is administered with a fat-rich meal. In patients with renal impairment (e.g. creatinine clearance ≧50 ml/min) or hepatic cirrhosis, the clearance of terbinafine is reduced by approximately 50%.

In the steady state, e.g. when the trough and peaks are constant after several days dosing, in comparison to the single dose, the peak terbinafine blood concentration (Cₘₐₓ) is 25% higher and the AUC increases by a factor of 2.5. This is consistent with an effective half-life for terbinafine of ca. 36 hours.

Pharmacokinetic and absorption properties have been disclosed in e.g. J. Faergemann et al. Acta Derm. Venereol. (Stockh.), 1997, 77, 74-76 and earlier articles. Little has been disclosed on steady-state pharmacokinetics and absorption on cessation of steady-state treatment.

The site of absorption of terbinafine is not known and as indicated above there is no clinically proven correlation of effect with pharmacokinetic profile so there is no rational starting point to provide a pharmaceutical composition containing terbinafine with improved therapeutical effects.

Despite the very major contribution which terbinafine has made, the reported occurrence of undesirable Adverse Events has been an impediment to its wider oral use or application. The particular difficulties encountered in relation to oral dosing with terbinafine have inevitably led to restrictions in the use of terbinafine therapy for the treatment of relatively less severe or endangering disease conditions, e.g. tinea pedis.

By the present invention there are provided novel terbinafine compositions, which meet or substantially reduce difficulties in terbinafine therapy hitherto encountered in the art. In particular it has been found that the compositions of the invention contain terbinafine in sufficiently high concentrations to permit convenient oral once-a-day administration, while at the same time achieving improved safety and tolerability in terms of fewer Adverse Events.

We have surprisingly found that terbinafine may be effectively dosed for a much shorter duration of time than previously contemplated. Thus the present invention enables reduction of terbinafine treatment times required to achieve effective therapy, reducing the exposure-time to terbinafine, and improving the global safety profile. In addition it permits closer standardization as well as optimization of on-going daily dosage requirements for individual subjects receiving terbinafine therapy as well as for groups of patients undergoing equivalent therapy.

By closer standardization of individual patient therapeutic regimens, dosaging parameters for particular patient groups, as well as monitoring requirements, may be reduced, thus substantially reducing the cost of therapy.

After exhaustive testing we have found that the tolerability of terbinafine may be significantly improved with immaterial loss in efficacy by administering compositions of terbinafine adapted to produce a reduced Cₘₐₓ and/or Cₘₐₓ/AUC ratio relative to immediate release formulations with the same dosage.

In one aspect the present invention provides an oral terbinafine sustained release pharmaceutical composition, hereinafter referred to as “a composition of the invention”.

In a further aspect, the present invention provides a method of administering terbinafine to a subject in need of terbinafine treatment which comprises administering to the subject a composition of the invention.

In a yet further aspect the present invention provides for the use of terbinafine as active agent in the manufacture of a composition of the invention.

In yet a further aspect the invention provides a pack containing a plurality of compositions of the invention arranged to be dispensed once a day for at least one week, preferably at least three weeks, and less than 10 weeks. Conveniently the treatment period is for 4 to 6 weeks in onychomycosis. This period represents the shortest treatment duration available to date for treating this chronic skin infection.

In another aspect the present invention provides an oral terbinafine sustained release pharmaceutical composition. Up to now there has not been any suggestion to use clinically such sustained release compositions, inter alia because terbinafine was conveniently administered effectively once a day.

It is surprising that terbinafine in the form of a composition of the invention is as effective as with immediate release compositions but exhibits fewer Adverse Events than expected.

For example, a composition releases terbinafine to the extent of 50% over a 120 minute period as measured by standard in vitro studies, e.g. in pH 3 using the paddle method.

For example, a composition releases terbinafine to the extent of 30 to 40% over a 60 minute period, to the extent of 40 to 50% over a 120 minutes period, to the extent of 40 to 60% over a 180 minutes period, to the extent of 45 to 65% over a period of 240 minutes, and to the extent of 50 to 70% over a period of 360 minutes, as measured by standard in vitro dissolution tests, e.g. in pH 3 using the paddle apparatus.

Suitable sustained release forms are described in Pharmazeutische Technologie, Thieme Verlag, Stuttgart/New York 2nd Edition 1991, Ed. H. Sucker, P. Fuchs, P. Spieser, e.g. p. 370-390. Further systems are described in e.g. Pharmaceutical Dosage Forms, Ed Herbert A. Lieberman, Leon Lachman, Joseph B. Schwartz, 2nd edition Vol 3, Marcel Dekker; and Remington: The Science and Practice of Pharmacy Ed Alfonso Gennaro, 19th edition, 1995.

A wide variation of sustained release systems may be used. Suitable sustained release formulations may operate by controlling the release of terbinafine by dissolution, diffusion, and preferably by osmotic pressure mechanisms.

Details of excipients of useful in the compositions of the invention are described in Fiedler, H. P. “Lexikon der Hilfsstoffe für Pharmazie, Kosmetik und angrenzende Gebiete”, Editio Cantor Verlag Aulendorf, Aulendorf, 4th revised and expanded edition (1996); “Handbook of Pharmaceutical Excipients”, 2nd Edition, Editors A. Wade and P. J. Weller (1994), Joint publication of American Pharmaceutical Association, Washington, USA and The Pharmaceutical Press, London, England; or may be obtained from brochures from the relevant manufacturers, the contents of which are hereby incorporated by reference.

Terbinafine may be used in free base form or in e.g. pharmaceutically acceptable form. Preferably the hydrochloride salt form is used.

The amount of terbinafine in composition of the invention will of course vary, e.g. depending on to what extent other components, are present.

In general, however, the terbinafine will be present in an amount within the range of from 0.1 to about 35% by weight based on the total weight of the composition.

Compositions will preferably be compounded in unit dosage form, e.g. by filling into capsule shells, e.g. soft or hard gelatine capsule shells or by tabletting or other moulding process.

Thus unit dosage compositions of the invention, suitable for administration once or twice daily (e.g. depending on the particular purpose of therapy, the phase of therapy etc.) will appropriately comprise half or the total daily dose contemplated. The compositions of the invention may be administered twice or three times a week. Preferably the compositions of the invention are administered once-a-day.

The pharmacokinetic properties of the compositions of the invention may be determined in standard animal and human pharmacological (bioavailability) trials.

For example one standard pharmacological trial may be carried out in healthy male or female non-smoking volunteers aged between 18 to 45 years having within 20% of the ideal body weight.

The trial may be a single dose crossover application. The subjects are domiciled for 24 hours.

Blood samples are taken for 1, 2, 4, 8, 16, 32 and 72 hours post administration of of a composition of the invention and tested for terbinafine. Terbinafine blood plasma concentrations may be determined in conventional manner, e.g. by HPLC or GLC analytical techniques. Safety is judged according to a standard checklist based on Adverse Event symptoms after 1 week.

Preferably the dose of terbinafine is 400, 600 or 700 mg per day. The safety of terbinafine at such a dose over the short duration of treatment is surprising. The compositions of the invention preferably exhibit a Cₘₐₓ 100-250%, e.g. 100-150%, of that shown by 250 mg immediate release Lamisil tablets, e.g. administered as a single dose and/or in the steady state, e.g. once a day for 7 days.

Preferably the C_{16 hour} (drug blood concentration 16 hours after administration) is greater than the C_{16 hour} observed with a 250 mg immediate release Lamisil tablet.

Preferably the composition of the invention is formulated so that the Tₘₐₓ appears 3 to 4 hours after administration.

Pharmacokinetic drug skin and nail concentration studies may be carried out according to the same principles as set out for the above-mentioned standard pharmacological trials. For example a clinical trial may be effected with daily dosing of compositions of the invention over a 3-week treatment period.

The compositions of the invention are useful for the same indications as for immediate release Lamisil tablets. The utility of compositions of the invention may be observed in standard clinical tests or standard animal models. For example, one can ascertain dosages of the compositions of the invention giving AUC blood levels of terbinafine equivalent to AUC blood levels giving a therapeutical effect on administration of known terbinafine oral dosage forms, e.g. a tablet, e.g. a matrix tablet, e.g. based on hydroxypropyl methylcellulose (HPMC), e.g. of a nominal viscosity (2% in water) of 100 000 mPas.

The compositions of the invention are particularly and surprisingly well tolerated with regard to the Adverse Events mentioned above, provoking fewer Adverse Events than would be expected on a simple multiple of the 250 mg immediate release Lamisil tablet. The compositions of the invention provoke fewer Adverse Events when coadministered with CYP2D6 substrates such as nortriptyline, desipramine, perphenazine, metoprolol, encainide and propafenone.

The compositions of the invention are particularly effective e.g. against onychomycosis.

From the clinical trials it is seen that the compositions of the present invention are just as efficacious particularly in aged patients, e.g of 70 years, and above, in patients with renal impairment (e.g. creatinine clearance ≧50 ml/min) or hepatic cirrhosis, and yet tend to provoke surprisingly fewer Adverse Events than expected for the dose given. Moreover the variation in AUC between fasted and fed state is less than expected. Preferably, there is no food effect.

A therapeutic clinical trial may be effected based on the principles of standard pharmacological trial mentioned above.

A randomized double-blind positive-controlled and placebo-controlled study may be effected with subjects having onychomycosis of the toe nail confirmed by microscopy and culture. Treatment is carried out over 12 weeks. Clinical trials may be effected in several hundred patients to ascertain the freedom from Adverse Events. However therapeutic efficacy may be shown in trials with 25 patients aged over 12 years.

Safety is evaluated by an Adverse Event report of clinical aspects and vital signs. Efficacy is determined by microscopy, culture procedures and visually looking at signs and symptoms. Efficacy is seen in patients with the fungi described above especially Trichophyton rubrum, Trichophyton mentagrophytes and Epidermophyton floccosum. Patients include those with predisposing factors such as impaired blood circulation, peripheral neuropathy, diabetes mellitus, damage from repeated minor trauma, and limited immune defects as well as AIDS. Patients have (i) distal lateral subungual onychomycosis starting at the hyponychium spreading proximally to the nail bed and matrix, (ii) and proximal subungual onychomycosis, wherein the fungus infects the cuticle and eponychium to reach the matrix where it becomes enclosed into the nail plate substance, (iii) total dystrophic onychomycosis, (iv) superficial white onychomycosis.

If desired serum concentrations of terbinafine may be evaluated in conventional manner or as described herein. Concentrations of terbinafine in the nail may be evaluated by both photo-acoustic spectroscopy and nail clipping followed by analysis, indicating presence of terbinafine in the nail-bed.

Clinical trials may be effected in particular sub-sets of subjects e.g. those with impaired renal or hepatic function.

The particular safety of compositions of the invention are shown in standard tolerability studies wherein terbinafine in immediate release form, such as a capsule are administered at dosages higher than normal. For example tolerability studies in beagle dogs may be effected peroral (p.o.) over 24 weeks at daily doses of from 60 to 300 mg/kg, e.g 120 mg/kg, animal body weight. Pharmacokinetic evaluations (toxicokinetics), e.g. Cₘₐₓ, AUC and Tₘₐₓ are measured. The following parameters are monitored: alanine aminotransferase, albumin, alkaline phosphatase, aspartate aminotransferase, calcium, chloride, total cholesterol, cholinesterase, creatine kinase, creatinine, glucose, inorganic phosphorus, magnesium, potassium, protein electrophoresis, sodium, total bilirubin, total protein, triglycerides, and urea as well as Glutamate dehydrogenase (GLDH), Lactate dehydrogenase (LDH) and LDH isoenzymes, and gamma glutamyltransferase (GGT).

Changes in the standard clinical chemistry parameters measured for liver dysfunction are lower than expected for the increased dose. We have also found surprisingly any such dysfunctions are transient and functional. This indicate the excellent tolerability of a composition of the invention e.g. with a Cₘₐₓ/AUC lower than for an immediate release form.

Following is a description by way of example only of compositions of this invention.

### EXAMPLE

| component | amount | |
|---|---|---|
| terbinafine hydrochloride | 675 | mg |
| HPMC (Methocel ® K100MP) | 95.4 | mg |
| microcrystalline cellulose | 180 | mg |
| colloidal silica (Aerosil 200) | 4.8 | mg |
| magnesium stearate | 4.8 | mg |
| total | 960 | mg |

The formulation is prepared by conventional manners. Terbinafine hydrochloride may be pre-granulated with e.g. 33.6 mg of the hydroxypropyl methylcellulose.

In a standard in vitro dissolution test with citrate buffer (pH 3) using the paddle method apparatus, the formulation of the example shows a release profile as described in Table 1.

TABLE 1

| | | | | | | | |
|---|---|---|---|---|---|---|---|
| time [min] | 0 | 60 | 120 | 180 | 240 | 360 | 720 |
| % dissolved | 0 | 35.6 | 44.2 | 50.9 | 56.2 | 63.9 | 77.3 |
| (mean, n = 3) | | | | | | | |

1. A terbinafine sustained release pharmaceutical composition for oral administration.

2. A method of administering terbinafine to a subject in need of terbinafine treatment which comprises administering to the subject a composition of claim 1.

3. The method of claim 2 wherein the composition is administered once a day.

4. The method of claim 3 wherein the composition is administered for 6 weeks.

5. The method of claim 4 wherein the subject suffers from onychomycosis.

6. Use of terbinafine as active agent in the manufacture of a composition according to claim 1.

7. Use acoording to claim 6 in a method according to claim 2, 3, 4, or 5.

8. A pack containing a plurality of compositions according to claim 1 arranged to be dispensed once a day for 6 weeks.
